(19) United States
(12) Patent Application Publication
(10) Pub. No.: US 2009/0260638 A1
(43) Pub. Date: Oct. 22, 2009

(54) RESTRAINT MITT

(76) Inventor: Ronald F. Duplessie, Waterford, MI (US)

Correspondence Address: DAVID L. KING, SR., 5131 N.E. COUNTY ROAD 340, HIGH SPRINGS, FL, 32643, US

(21) Appl. No.: 12/423,305

(22) Filed: Apr. 14, 2009

Related U.S. Application Data
(60) Provisional application No. 61/046,819, filed on Apr. 22, 2008.

(51) Int. Cl. A61F 5/37; A41D 19/01

(52) U.S. Cl. 128/879; 2/158

(57) ABSTRACT

A thumbless mitt 10 for receiving a hand 2 has a cloth outer shell cover 12, a cloth inner liner 14 and a non-pliable palm center 20 interposed between the inner liner 14 and the outer shell cover 12 on a palm facing portion 18 of the mitt 10. The cloth inner liner 14 defines an inner chamber 19 into which the entire hand 2 is inserted. The non-pliable palm center 20 extends from the palm location to adjacent and along the fingers location, preferably centered on the palm side 18 of the mitt 10 and having a width sufficient to block at least two middle fingers from bending in a grasping position beyond the palm center 20. Two pairs of straps 30 and 40 are strategically located to prevent removal and or rotation of the hand 2inside the mitt 10.

### RELATED APPLICATIONS

The present invention claims benefit of priority to U.S. Provisional Patent Application Ser. No. 61/046,819 filed Apr. 22, 2008, the disclosure of which is incorporated herein by reference in its entirety.

### TECHNICAL FIELD

The present invention relates to a patent safety device to prevent the removal of catheters, intravenous lines and gastro feeding tubes.

### BACKGROUND OF THE INVENTION

Mitt restraints are making a positive impact in physical therapy. Stroke is the leading cause of adult disability in the United States (Stroke is the leading cause of adult disability in the United States, 2008). A new method of physical therapy known as Modified Constraint Induced Movement Therapy described in The Journal of the American Medical Association 2006, article “Effect of Constraint-Induced Movement Therapy on Upper Extremity Function 3 to 9 Months After Stroke” 296 (17), pages 2095-2104 by Wolf, S. L., Winstein, C. J., Miller, J. P., Taub, E., Uswatte, G., Morris, D., Giuliani, C., Light, K. E., Nichols-Larsen, D. uses mitt restraints (mechanical restraints) with clients who have sustained a brain attack (stroke).This new therapy has resulted in significant improvement in regaining upper extremity function whereas, traditional physical therapy was unsuccessful. Mitt restraints are used as an asset in this venue.

Regarding hospitals and nursing homes, the use of mechanical restraints and restraint alternatives has been controversial since the 1990s. “The right to be free of unnecessary and inappropriate physical and chemical restraints”, stated by E. H. Turnham (n.d.) in the Federal Nursing Home Reform Act from the Omnibus Budget Reconciliation Act of 1987, paragraph 5 is a right that all individuals embrace. It is encouraging that restraint alternative measures have been successful for the majority of hospital and nursing home populations. However, there is still a minority that restraint alternatives fail. Diane Lancaster, Director of Quality Measurement and Improvement for Nursing at Brigham and Women's Hospital stated, “An average of 15 patients [5%] are restrained at the hospital on any given day . . . ” from a 2007 BWH (Brigham and Women's Hospital) Journal article by L. F. Rodriguez, Patient Safety Focus: Use of restraints, paragraph 2. The most commonly used restraints in the context of this invention are soft wrist restraints and bulky mitts that preclude finger movement. Some other mechanical restraints include: vest, straps across a body, four-point (soft wrist plus ankle restraints), Geri Chair, straight jacket, Lap Buddy for a wheelchair, and bed enclosure to prevent wandering.

Generally accepted professional practices dictate that restraints may only be used when it is clinically justified. Such justifications include situations where restraint is necessary: (i) to facilitate appropriately the provision of medical care; (ii) to control a resident's unanticipated violent or aggressive behavior that places either the resident or others in imminent danger; or (iii) as a last resort to provide safety when all other less restrictive methods have been attempted and failed as stated by A. R. Acosta, 2004, paragraph 3, of the findings of Assistant Attorney General, western district of Arkansas, R. Alexander Acosta regarding the Baxter Manor Nursing Home.

Disoriented and confused patients can cause severe injury to self if not immediately interrupted in their attempts. In cases where on-site personnel are not available in the rooms to intervene, patients can remove a line and hemorrhage or pull out life-saving equipment and die. Hence, cases pertaining to: small children, disoriented or confused patients of any age attributed to medication or other reasons, older people with dementia or Alzheimer's, and the mentally ill, are at high risk or have a tendency to remove lines and tubes. Utilizing the least restrictive mechanical device to protect the patient becomes necessary when the alternatives have failed and when confusion is to the degree that patients can no longer make safe decisions.

In 2007, A BWH (Brigham and Women's Hospital) Journal article by L. F. Rodriguez, Patient Safety Focus: Use of restraints; states in paragraph 5, “The restraint taskforce has reviewed many new products, such as hand mitts to help prevent patients from pulling at IVs and sleeves that go over the arms to disguise dressings”. Indeed, the medical profession is very aware that to protect some patients from unintentional self-harm mechanical restraints are still necessary to protect life.

Even though mechanical restraints have had a positive impact to protect those from unintentional self-harm—from the person being strapped onto a gurney and placed in an ambulance to the hospitalized medically ill or trauma patient, A. R. Acosta, Assistant Attorney General of Arkansas points out “improper use of restraints [has also had] disastrous consequences, including loss of function, depression, falls and injuries, loss of dignity, weight loss, pressure sores, serious injury, and even death” in paragraph 6 of the findings of Assistant Attorney General, western district of Arkansas, R. Alexander Acosta regarding the Baxter Manor Nursing Home. Articles have noted these occurrences from side rails to seclusion rooms. Therefore, increasing awareness through education regarding the danger of misuse of any type of restraint could prevent deaths and injuries.

In 1987, The United States Federal Government concerned with misuse of restraints and other issues created the Omnibus Budget Reconciliation Act of 1987 (OBRA) by Turnham, E. H. (n.d.). In short, this federal document became known as The Nursing Home Reform Act of 1987.

OBRA held nursing homes accountable for: staff competency, supplying enough staff, and having all residents under a doctor's care. Most importantly, nursing homes were mandated to find ways to reduce the physical restraints used on their residents with the primary goal to be restraint free and had nursing homes looking for alternatives to comply with federal regulations. A few years later, the Joint Commission on Accreditation of Healthcare Organizations (JCAHO) a highly regarded nonprofit organization that commends good patient quality care by giving facilities accreditation status created general guidelines for hospitals taken from, Patient rights and organization ethics, page 44: Intent of TX.7.5. Critical Access Hospital COPs and corresponding JCAHO AMH standards. Retrieved Jan. 12, 2008, from Iowa Hospital Association Web site: http://www.ihaonline.org/cah/cops.pdf

Today, in many cases the patient is provided home care as an alternative to a nursing home or a hospital. In these cases the care giver is often the spouse or an adult child who is given the necessary instructions to provide care along with working under the direction of a visiting nurse. In these situations when their loved one is bedbound and confused and fed by a gastric feeding tube and/or on a ventilator, instead of having to resort to big, bulky, intimidating mitt restraints or soft wrist restraints as the nursing homes and hospitals are predominately using in these types of situations, it seems that an improved method of restraint that is less severe yet effective is needed.

The present invention provides this type of solution. It permits the arms to be mobile yet prevents the hands from being able to grasp objects.

### SUMMARY OF THE INVENTION

A thumbless mitt for receiving a hand has a cloth outer shell cover, a cloth inner liner and a non-pliable palm center interposed between the inner liner and the outer shell cover on a palm facing portion of the mitt. The cloth inner liner defines an inner chamber into which the entire hand is inserted. The non-pliable palm center extends from the palm location to adjacent and along the fingers location, preferably centered on the palm side of the mitt and having a width sufficient to block at least two middle fingers from bending in a grasping position beyond the palm center.

The cloth outer shell cover and the cloth inner liner are preferably sewn or stitched together along seam edges and turned inside out to conceal the seam edges. The non-pliable palm center is preferably made of plastic and is a flat rectangle which is secured by stitching together the inner liner and outer shell cover around the perimeter of palm center.

The mitt preferably has two pairs of straps, a first strap pair extending from a sleeve portion of the mitt at or below the wrist and a second strap pair attached to the outer shell cover on a side opposite the non-pliable palm center. The second strap pair is adjacent to the back side portion of the mitt contacting the back of the hand, between the knuckles location and above the location of the wrist so when tightened, the second strap brings the palm of the hand against the location of the non-pliable palm center. Each strap is preferably made of flat cloth cord and has one or more buckles for tightening the straps. The first strap pair at the wrist location has one long strap for wrapping completely around a wrist in the sleeve portion of the mitt. The entire mitt is machine washable and reusable.

### DEFINITIONS

Ankle restraint secures each foot toward the side and near the foot of a bed. Description is the same as a soft wrist restraint.

Bed Enclosure an enclosed zippered mesh canopy over a hospital bed that allows freedom of movement. It is used to deter a confused person from getting out of bed unassisted to prevent falls. The bed enclosure is not intended for an agitated person or a person with life sustaining equipment.

Geri Chair a vinyl covered reclining chair on wheels. When it is in a reclining position or has a feeding tray attached to the arms of the chair, it is classified as a restraint.

IV or intravenous instilling fluids or medication into a vein via a syringe or needle that is attached to thin tubing, which is connected to a small or large plastic bag containing fluid or medication.

Lap Buddy a thick foam covered vinyl tray that fits a wheelchair. It has extensions that press into the opening between the side panels of the wheelchair and the armrests.

Mechanical restraint a manufactured product that restricts bodily movement, such as: chair restraints, mittens, wrist and ankle restraints, straight jackets, enclosed beds, bed side rails, arm splints, and vest restraints.

Mitt restraint any type of fabric, foam, or plastic covering the hand, whether immobilizing or allowing finger and thumb movement, along with securing the covering at the wrist with a shoe lace, strap, or belt, to prevent removal. A common example would be boxing gloves. A child who has winter mittens on and can not remove them is also classified as a mitt restraint.

Patient refers to a person admitted into a hospital.

Physical restraint classified two-fold. First, it is restraining a person with bodily force. Second, it is a general term including all restraints most commonly used in hospitals and nursing homes. These include: chair restraints, mittens, wrist and ankle restraints, straight jackets, enclosed beds, bed side rails, arm splints, and vest restraints.

Resident refers to a person admitted into a nursing home.

Soft wrist restraint inner soft foam adhered to fabric that resembles a thick bracelet. Straps are attached to the bracelet so the straps can be tied with a slip knot (for easy release) to the bed frame to keep the arms apart.

Vest restraint applied over the head and covers the front and back torso. Two long straps on each side secure the front and back panels and each set of straps are tied with a slip knot (for easy release) to the bed frame.

### BRIEF DESCRIPTION OF THE DRAWINGS

These and further objects of the present invention will be more fully understood from the following description of the invention reference to the illustrations appended hereto:

FIG. 1 illustrates a perspective view of the mitt according to the present invention.

FIG. 2 shows a top plan view of the mitt of FIG. 1.

FIG. 3 shows a first side plan view of the mitt made according to the present invention.

FIG. 4 shows a second side plan view of the mitt made according to the present invention.

FIG. 5 shows a bottom or palm side plan view of the mitt made according to the present invention.

FIG. 6 is a closed end view of the mitt made according to the present invention.

FIG. 7 is an open end view of the mitt made according to the present invention.

FIG. 8 shows a cross sectional view of the mitt made according to the present invention prior to cinching the straps.

FIG. 8A shows a cross sectional view of the mitt made according to the present invention after cinching the straps.

### DETAILED DESCRIPTION OF THE INVENTION

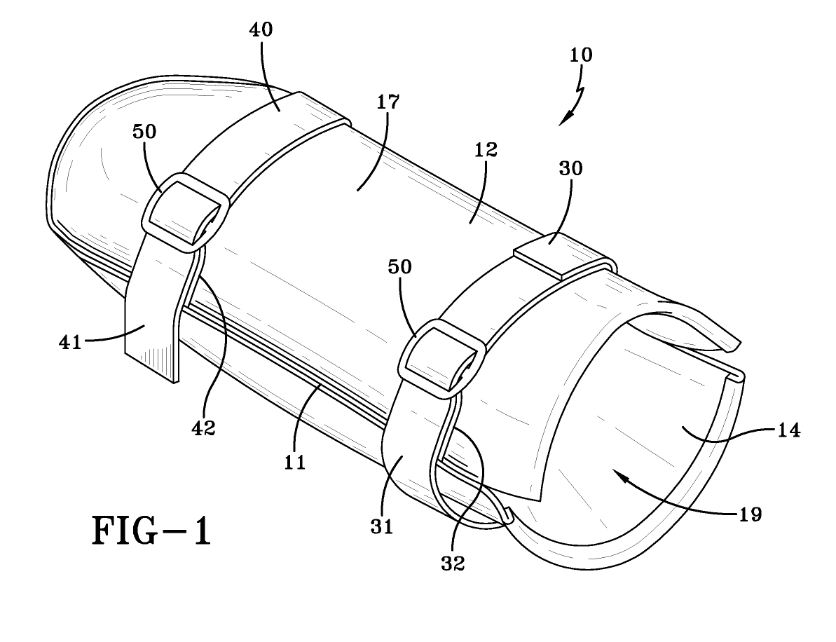

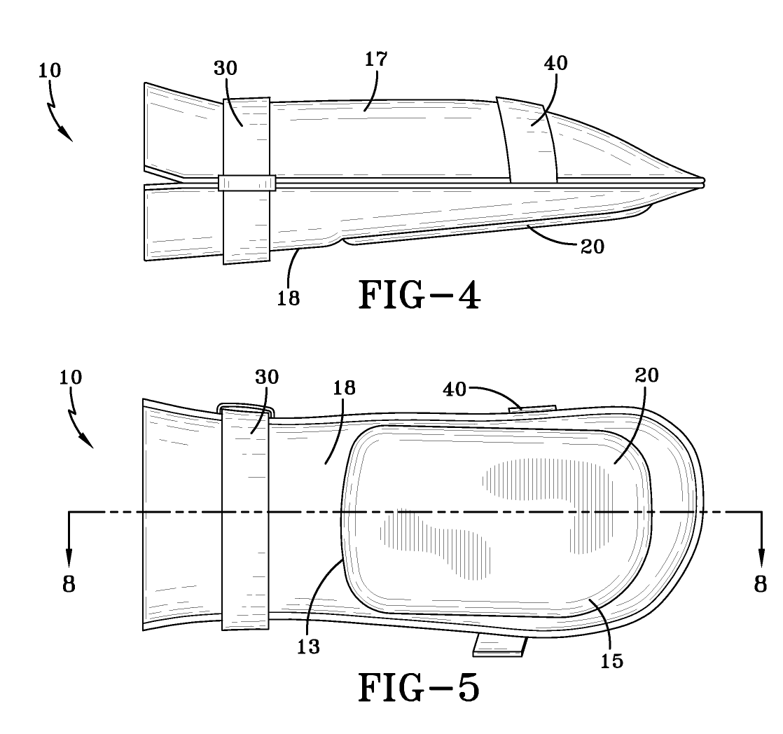

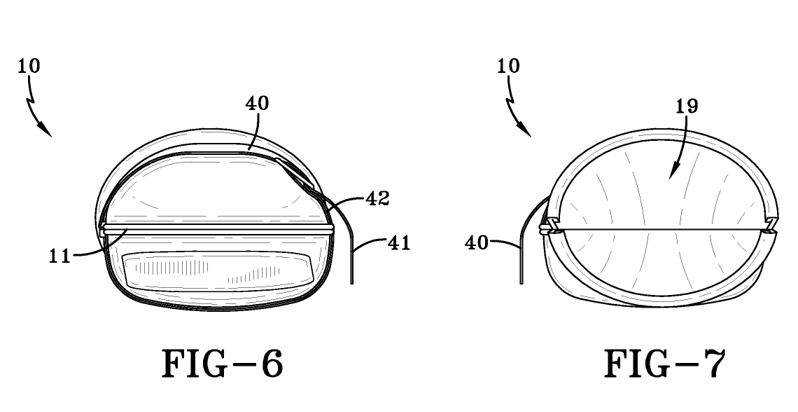

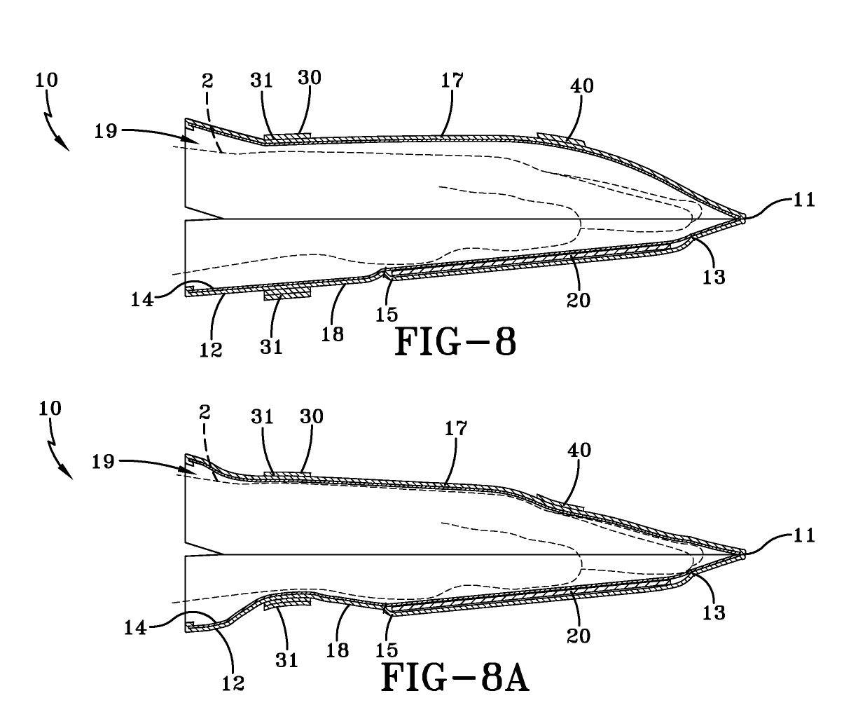

With reference to FIGS. 1-5, a perspective view of the thumbless mitt for receiving a hand is shown. As shown in FIGS. 3, 4 and 5, the thumbless mitt 10 has a cloth outer shell cover 12, a cloth inner liner 14 and a non-pliable palm center 20 interposed between the inner liner 14 and the outer shell cover 12 on a palm facing portion 18 of the mitt 10. As shown in FIGS. 8 and 8A, the cloth inner liner 14 defines an inner chamber 19 into which the entire hand 2 shown in dashed lines is inserted. The non-pliable palm center 20 extends from a palm location of the mitt 10 to an adjacent and along the fingers location of the mitt 10. Preferably, the palm center 20 is positioned centered on the palm side 18 of the mitt 10 and having a sufficient width to block at least two middle fingers from bending in a grasping position beyond the palm center 20.

Along the edges of the cloth outer shell cover 12 and the cloth inner liner 14 are seam edges 11. The seam edges 11 are preferably sewn or stitched together and once sewn or stitched together the entire assembly is then turned inside out to conceal the seam edges 11 from exterior of the mitt 10.

As shown in the cross sectional view of FIG. 8 and also in FIG. 8A showing the palm portion 18 of the mitt 10 is the non-pliable palm center 20 which is preferably made of a non-pliable semi-rigid plastic that is sufficiently thick that the wearer cannot bend or manipulate the palm center 20. The palm center 20 as shown in FIGS. 8 and 8A, is interposed between the inner liner 14 and the outer shell cover 12 and is then as shown in FIG. 5, stitched along the perimeter edges 15 of the palm center 20 such that the entire assembly is located preferably centered on the palm portion 18 of the mitt 10. As shown the stitching around the palm center 20 is identified as stitched edges 13, preferably the palm center 20 has a cross sectional thickness that is sufficient to resist bending by the wearer. As shown the cross sectional thickness is between 90 thousandths and ⅛^{th} of an inch, however, thicker or thinner materials can be used depending on the strength of the materials considered. One important aspect of the invention is that the mitt 10 is completely washable, as such it is preferred that the non-pliable palm center 20 be made of a material that can withstand repeated washings. Plastic is an ideal material for such an application. Preferably a polyethylene type material or other material of sufficient durability to withstand repeated cycles of hot water and drying cycles within washing and drying equipment.

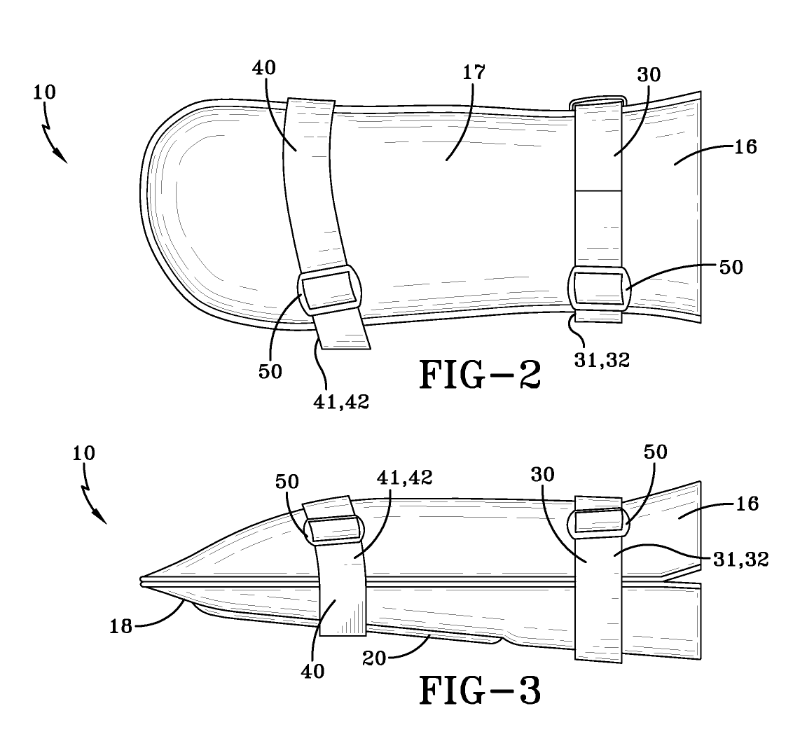

With reference to FIG. 5, the bottom or palm side of the mitt 10 is illustrated showing the palm center 20. Flipping the entire mitt 10 around 180 degrees the rear side of the mitt is shown in FIGS. 2, 8 and 8A wherein two pairs of straps 30 and 40 are shown. The first strap pair 30 extends from a sleeve portion 16 of the mitt 10 at or below the wrist of the hand 2 as shown in phantom lines. A second pair of straps 40 is attached to the outer shell cover 12 on a side opposite the non-pliable palm center 20 on the back side portion 17 of the mitt 10. This back side portion 17 of the mitt 10 is the portion wherein the back of the hand 2 would be positioned inside the chamber 19 formed by the inner liner 14. The strap pair 40 preferably is located between the knuckles location of the fingers and above the wrist location so that when tightened the second strap 40 brings the palm and fingers of the hand against the location of the non-pliable palm center 20. The strap pair 30 has a strap 31 and a strap 32. Preferably the strap 31 is a long strap which can be wrapped completely about the wrist around the sleeve portion 16 of the mitt 10. As such when attaching the mitt 10 over a patient's hand, as illustrated, the strap 31 is wrapped and then attached to the strap 30 using one or more buckles 50 as shown in FIGS. 1-3. The buckles 50 are a metallic material and are sufficient to make it difficult for a patient to unlatch or otherwise release the straps 31, 32 from the tightened position. As shown this first strap pair 30 prevents the mitt 10 from being withdrawn back over the hand 2. The wrist being much smaller than the hand portion makes it virtually impossible for the patient to withdraw the hand from the mitt 10 once it is attached in this fashion. As shown in FIGS. 1-3, the second strap pair 40 has straps 41 and 42 as indicated, located near the knuckles of the fingers and above the wrist. It is important these straps 41, 42 be strategically located in this position such that when tightened the second strap pair 40 brings the palm of the hand in close proximity to the non-pliable palm center 20 as the strap 41 is tightened, the hand is put into close proximity to the palm center 20. The palm center 20 has a width sufficient to cover at least two of the middle fingers of the hand. The width can be adjusted wider, but preferably not much smaller, in this fashion it is virtually impossible for the patient to grasp anything due to the extension of the non-pliable palm center 20 from the palm location outwards along and adjacent to the fingers that extend inside the mitt 10 as illustrated. This feature has been found essential because patients while they cannot remove the mitt 10 due to the first strap pair 30, it has been determined that the patient can rotate the hand 2 inside the mitt 10 and as such the back side of the mitt 17 would be sufficiently pliable that the patient can grasp even while wearing the mitt 10.

By providing the second strap pair 40 as illustrated in FIG. 8A, the hand 2 is brought into close proximity to the non-pliable palm center 20 and as such the hand cannot be sufficiently cupped to grasp anything nor can the hand be rotated inside the mitt 10. These features are extremely important in that the purpose of the mitt is to prevent the patient from being able to grasp any of the lines or feeding tubes in such a fashion that they can remove them and inadvertently cause harm to themselves. By providing the mitt 10 as shown, the hand 2 is securely positioned inside the mitt 10 by the first strap pair 30 and prevented from any rotation by the second strap pair 40. This results in a secure positioning of the hand. Again the second strap pair 40 employing straps 41 and 42 is secured by the use of one or more buckles 50 as illustrated in FIGS. 1-3.

As shown each strap 31, 32, 41 and 42 are preferably made of cloth cord material and as such are also machine washable, making the mitt 10 entirely reusable for more than one patient. This helps reduce the cost of the device while providing a superior means of preventing the patient from withdrawing lines or feeding tubes. As shown one of the advantages of the present mitt 10 is that the fingers and thumb are all moveable from inside the mitt. In other words they can be flexed and moved and no restraining feature is provided that could injure the patient. This mobility of the fingers and thumb is important for the comfort of the patient in that he can feel his fingers and thumb, move his fingers and thumb and feel reasonable comfortable while wearing the mitt 10. The mitt 10 further is very non-obtrusive, it is provided in such a fashion that the inner liner 14 is a soft comfortable material, preferably cotton or a cotton blend, that is very breathable and therefore the hand does not sweat excessively wearing the mitt 10. Additionally, the outer cover 12 is made of a similar breathable material so that sufficient air circulation is occurring at all times. This is a huge advantage for the patient wearing such a device. It is important that the patient feel very comfortable while wearing the mitt 10, otherwise the patient feels constrained and the mitt 10 could create problems from a psychological perspective of the wearer. By providing a very comfortable fitting and wearing mitt 10 that is breathable preferably through the use of cotton material, the wearer does not feel overburdened by large, bulky mitts that are currently available.

Many of the mitts currently are provided with extremely large padding such that the hand is encumbered within a large almost boxer type mitt that creates a large unsightly bulbous effect around the hand. This is completely avoided with the present mitt 10 in that it is very lightweight, very comfortable in both its wear and its breathability such that the patient is not over encumbered nor feels burdened by the wearing of the mitt.

While the psychological factors of wearing such a mitt may seem trivial, to the patient under long term supervision these factors are critical in whether or not patients are going to be willing to wear such a device. These have been thought through very well in the present invention and overcome many of the serious problems with other forms of more obtrusive restraints. While it is recognized that the use of mitts and other restraints are not preferred. The most preferred method of caring for a patient is to provide a person within the room for 100 percent direct supervision of the patient. It is not always to provide this type of service, as such in a preferred alternative, the present inventors feel that the use of the present mitt is an excellent compromise wherein the patients safety is established by wearing the mitt 10 and the health care providers can be confident the mitt 10 is the least obtrusive way in which protecting the patient can occur. Even under the best supervision with 100 percent surveillance of the patient, it is not always possible to prevent many patients from injuring themselves by removal of their feeding tubes or IV lines, as such it is believed in those cases that the use of the mitt 10 of the present invention is the best alternative solution under these circumstances. As shown the mitt 10 is provided as a machine washable, reusable device that can be repeatedly used on a variety of patients. Alternatively, lower cost synthetic materials can be used that are completely disposable if so desired. It is important to understand the various alterations to the mitt can occur and still be within the scope of the invention. For example, although not shown, the back side 17 of the mitt 10 can be provided with an access port to provide attachment of an intravenous tube or line if needed. Additionally the back side 17 of the mitt 10 can be provided with a screen mesh, such that an observation of the patient's skin can occur easily. These and other alterations are well within the scope of the invention without altering the present inventive concept of providing a thumbless restraint mitt 10 in the absence of any thumb portion such that the hand is maintained in a non-grasping fashion that is impossible to rotate from within the inside of the mitt 10. These and other features heretofore have not been provided in other mitts or gloves used for such purposes.

Variations in the present invention are possible in light of the description of it provided herein. While certain representative embodiments and details have been shown for the purpose of illustrating the subject invention, it will be apparent to those skilled in this art that various changes and modifications can be made therein without departing from the scope of the subject invention. It is, therefore, to be understood that changes can be made in the particular embodiments described which will be within the full intended scope of the invention as defined by the following appended claims.

1. A thumbless hand mitt comprises:
a soft cloth mitt for receiving a hand, the soft cloth mitt having a cloth inner liner defining an inner chamber in which the entire hand is inserted, an outer shell cover covering the inner liner, and a non-pliable palm center interposed between the inner liner and outer shell cover on a palm facing portion of the mitt, the non-pliable palm center extending from the palm location outwardly to adjacent and along the fingers location.

2. The thumbless hand mitt of claim 1 wherein the inner liner and outer shell cover are stitched together to form the mitt.

3. The thumbless hand mitt of claim 1 wherein the mitt has a sleeve portion extending to cover at least a portion of the arm at or beyond the wearers wrist, the sleeve portion being open to the inner chamber.

4. The thumbless mitt of claim 1 has two pairs of straps, a first strap pair being attached to the sleeve portion at or below the wrist, a second strap pair attached to the outer shell cover on a side opposite the non-pliable palm center and adjacent to mitt portion covering the back of a hand, the second strap being positioned near location of the knuckles of the fingers and above the location of the wrist and when cinched brings the palm of the hand against the location of the non-pliable palm center.

5. The thumbless mitt of claim 1 wherein each strap pair is made of cloth and has one or more buckles for securing the straps.

6. The thumbless mitt of claim 5 wherein the first strap pair has a long strap for wrapping around the wrist in the sleeve portion of the mitt.

7. The thumbless mitt of claim 1 wherein the non-pliable palm center is centered on the palm side of the mitt.

8. The thumbless mitt of claim 7 wherein the non-pliable palm center has a width sufficient to block at least the two middle fingers from bending beyond the palm center in a grasping position.

9. The thumbless mitt of claim 8 wherein when secured on the hand the fingers and thumb are free to move, wiggle and flex, but not free to grasp objects.
